(19) United States
(12) Patent Application Publication
(10) Pub. No.: US 2018/0078815 A1
(43) Pub. Date: Mar. 22, 2018

(54) TREADMILL AND MONITORING SYSTEM

(71) Applicant: FOXCONN INTERCONNECT TECHNOLOGY LIMITED, Grand Cayman (KY)

(72) Inventors: CHANG-WEI KUO, New Taipei (TW); SU-NUNG CHAO, New Taipei (TW); YI-CHUNG TENG, New Taipei (TW); JUI-KUANG CHUNG, New Taipei (TW)

(21) Appl. No.: 15/708,047

(22) Filed: Sep. 18, 2017

(30) Foreign Application Priority Data
Sep. 18, 2016 (CN) 201621059378.X

(51) Int. Cl. A63B 22/02; A63B 24/00; G06F 19/00; A61B 5/0245

(52) U.S. Cl. CPC A63B 22/0242; A63B 24/0062; G06F 19/34; A63B 22/0023; A63B 24/0087; A63B 2230/04; A61B 5/0245

(57) ABSTRACT

A monitoring and computing system for use with a treadmill, includes an intelligent sensing and computing system includes the data collection box and the sensors. The sensors includes the infrared sensor for sensing the speed, the six axes sensor for sensing the slope and the step frequency, the electrocardiogram sensor for sensing the heartbeat. The aforementioned sensors and the data collection box are communicated with each other either by USB wire or blue tooth wireless

### BACKGROUND OF THE INVENTION

### 1. Field of the Invention

The present invention relates to a treadmill and the monitoring/computing system thereof, and particularly to the treadmill equipped with the intelligent sensing and computing device and the associated monitoring/computing system.

### 2. Description of Related Art

The modern treadmills are provided with the intelligent arrangement and the sensor equipments so as to adjust the speed and the slope of the walking track and sensing and monitoring the user's heartbeat to show the electrocardiogram which may be uploaded to the cloud network system for the user review. Most of time, the collected data are isolated and can not be efficiently used by the gymnasium management for either coaching the user for better exercises or managing the treadmills for better serving.

### SUMMARY OF THE INVENTION

The invention is to provide the treadmill and the monitoring and computing system thereof wherein the monitoring computing system may manage the different sport indexes/data to help the user understand his body situation and arrange the future plan. The system includes an intelligent sensing and computing system includes the data collection box and the sensors. The sensors includes the infrared sensor for sensing the speed, the six axes sensor for sensing the slope and the step frequency, the electrocardiogram sensor for sensing the heartbeat. The aforementioned sensors and the data collection box are communicated with each other either by USB wire or blue tooth wireless.

The monitoring/computing system includes the gateway, the wireless RF identification system, Wi-Fi wireless network base and the server system. The Wi-Fi wireless network station is connected with the server system, the wireless RF identification system is connected with the Wi-Fi wireless network station, the gateway and the data collection box are connected via the personal area network while the gateway and the Wi-Fi wireless network station mutually communicate with each other via the local area network or wide area network.

The data collection box sends the data derived from the sensors to the gateway via blue tooth, the gateway transmits the data to the Wi-Fi wireless network station and further to the server system for use of the browser or the application program of the mobile device.

The RF identification system includes a RF tag and a card reader wherein the RF tag corresponds to the user and the card reader corresponds to the individual treadmill.

The monitoring/computing system further includes an intelligent power wherein the gateway and the intelligent power are wirelessly connected with each other via ZigBee. When the user uses the card reader to read the personal RF tag, the wireless RF identification system may transmit the data to the Wi-Fi wireless network station, the data is further transmitted to the gateway via the Wi-Fi wireless station and the gateway may activate the intelligent power via ZigBee to have the treadmill ready in use.

The invention may be implemented via another way as described below. The treadmill includes a main body and an intelligent sensing and computing system which includes a data collection box and the treadmill sensors. The sensors includes the infrared sensor for sensing the speed, the six axes sensor for sensing the slope and the step frequency, the electrocardiogram sensor for sensing the heartbeat. The aforementioned sensors and the data collection box are communicated with each other either by USB wire or blue tooth wireless. The data collection box transmits the data to the gateway via the personal area network.

The data collection box includes a power key for on/off usage, a Micro USB connection port for charging, three USB connection ports and the printed circuit board assembly. The USB connection ports may connect to the individual sensors for obtaining the respective data.

The infrared sensor includes a first printed circuit board and a infrared reflection module, a first Micro USB connection port, a bottom cover for receiving the first printed circuit board, a top cover and the exterior mounting securing module wherein the first Micro USB connection port is connected to the USB connection port of the data collection box via the Micro/USB wire adaptor.

The six axes sensor includes a second printed circuit board, a processing module, a second Micro USB connection port, a top cover and a bottom cover, the processing module includes the accelerometer and the gyroscope wherein the second Micro USB connection port is connected to the USB connection port of the data collection box via the Micro/USB wire adaptor.

The electrocardiogram sensor includes electrode plates and wires connected thereto wherein the electrode plates are mounted upon the handles having the metal parts thereon so as to sense the minor signals and transmit such signals to the data collection box.

Compared with the traditional device, in the invention the intelligent sensing and computing system may be applied with different brand treadmills to send the different data or index, which is sensed and stored in the data collection box, to the gateway, and the gateway successively transmits such data to the Wi-Fi wireless network station and further to the server system for reviewing by the browser or the application program, thus not only integrally managing the data but also raising the managing level of the monitoring/computing.

### BRIEF DESCRIPTION OF THE DRAWINGS

FIG. 1 is a flow chart of the treadmill monitoring/computing.

FIG. 2 is the partial flow chart of FIG. 1 when the user is approaching the treadmill;

FIG. 3 is the partial flow chart of FIG. 1 when the intelligent power is on;

FIG. 4 is the partial flow chart of FIG. 1 during sensing the room circumstance;

FIG. 5 is the partial flow chart of FIG. 1 when the user is o-ting the treadmill;

FIG. 6 is a perspective view of the treadmill to which the treadmill system of FIG. 1 is applied;

FIG. 7 is an assembled perspective view of the data collection box of the treadmill of FIG. 6.

FIG. 8 is an exploded perspective view of the data collection box of the treadmill of FIG. 7;

FIG. 9 is another exploded perspective view of the data collection box of the treadmill of FIG. 8;

FIG. 10 is an assembled perspective view of the infrared sensor of the treadmill of FIG. 6;

FIG. 11 is another assembled perspective view of the infrared sensor of the treadmill of FIG. 10;

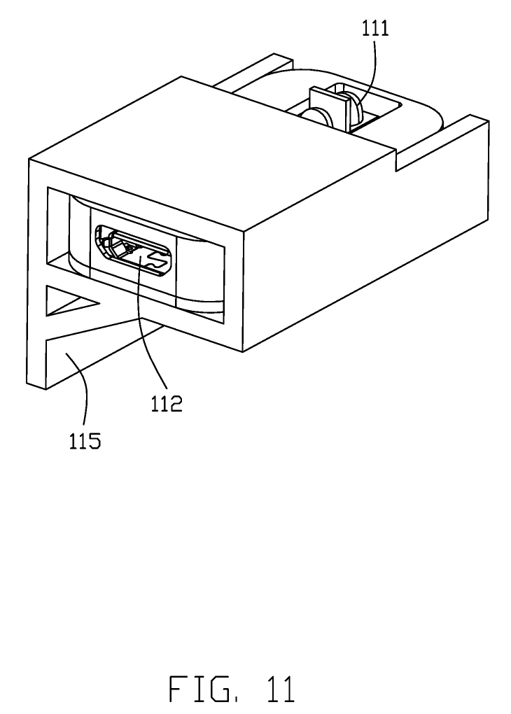

FIG. 12 is a partial exploded perspective view of the infrared sensor of the treadmill of FIG. 10;

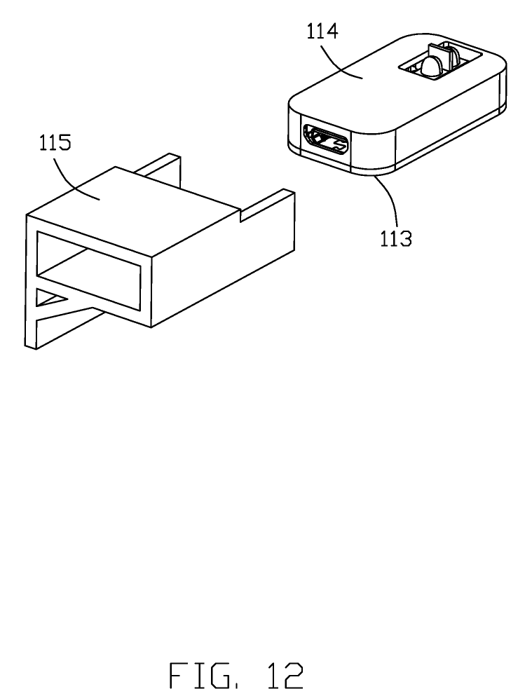

FIG. 13 is an exploded perspective view of the infrared sensor of FIG. 10 without the mounting securing module thereon;

FIG. 14 is a perspective view of the six axes sensor of the treadmill of FIG. 6; and

FIG. 15 is an exploded perspective view of the six axes sensor of the treadmill of FIG. 14.

### DETAILED DESCRIPTION OF THE PREFERRED EMBODIMENT

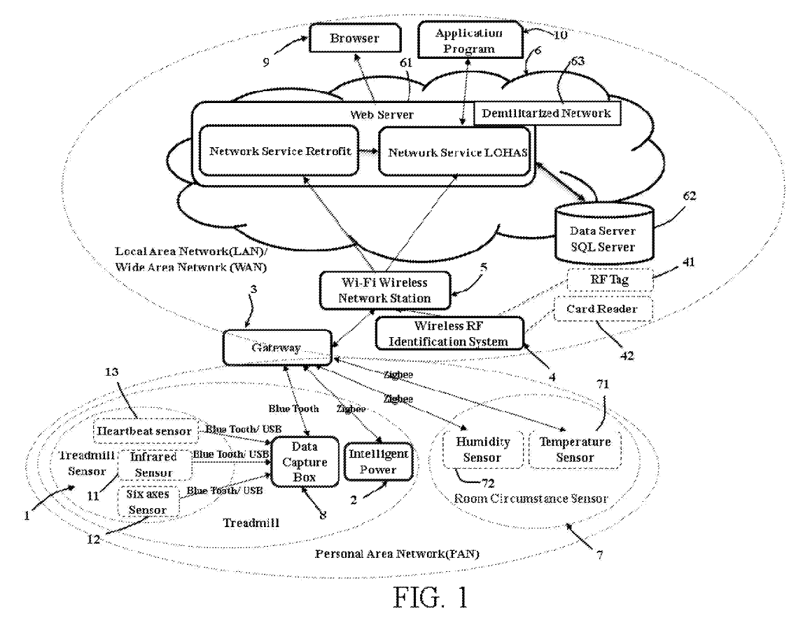

FIGS. 1-5 show the monitoring/computing for use with a treadmill. The system includes an intelligent sensing and computing system (not labeled), a gateway 3, an RFID (Radio Frequency Identification) 4, an intelligent power 2, a room circumstance sensor 7, a Wi-Fi wireless network station 5, and the server system 6. The Wi-Fi wireless network station 5 and the server system 6 are connected to each other, the RFID 4 and the Wi-Fi wireless network station 5 are connected to each other, the gateway 3 and the intelligent sensing and computing system are connected via the personal area network (PAN), and the gateway 3 and the Wi-Fi wireless network station 5 are mutually connected via the local area network or the wide area network.

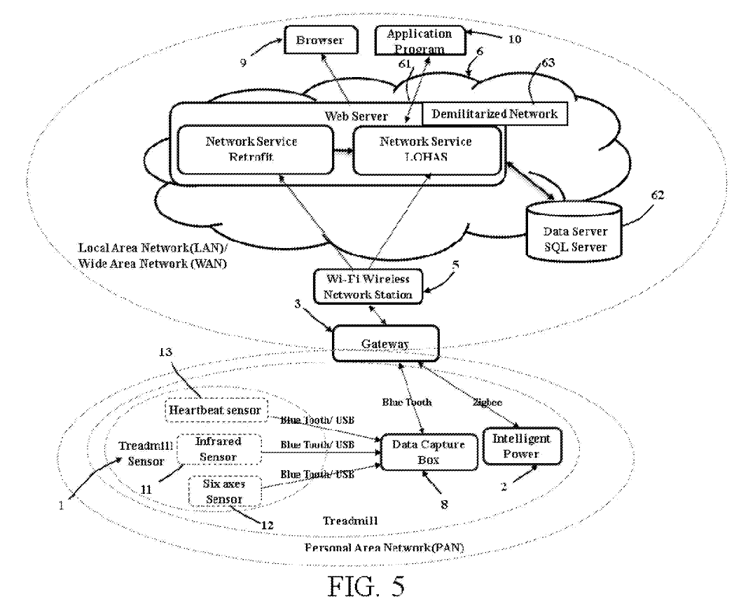

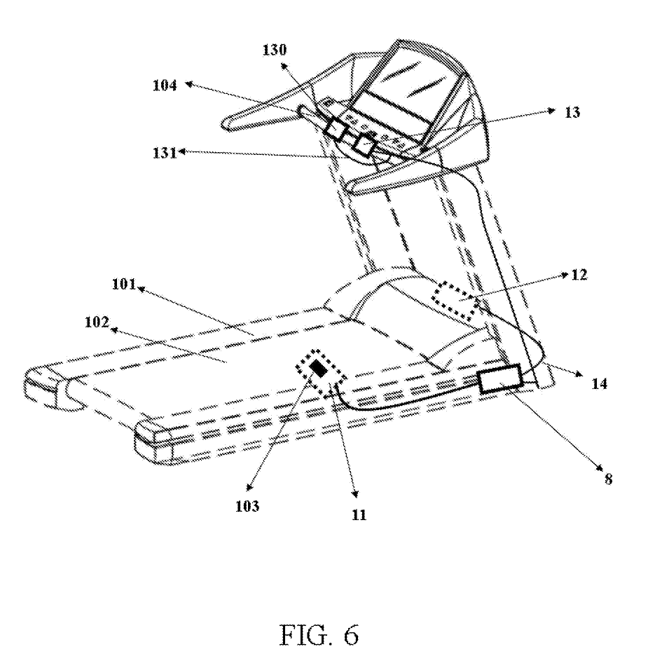

As shown in FIGS. 5 and 6, the intelligent sensing and computing system includes a data collection box 8 and the sensors 1 including the infrared sensor 11 for the speed, the six axes sensor 12 for sensing the slope and the step frequency, the electrocardiogram sensor 13 for sensing the heartbeat. In this embodiment, the sensor 1 is connected to the data collection box 8 for the signal transmission via the USB (Universal Serial Bus) wire 14. In other embodiment, the signals may be transmitted by the wireless personal area network (WPAN), e.g., the blue tooth.

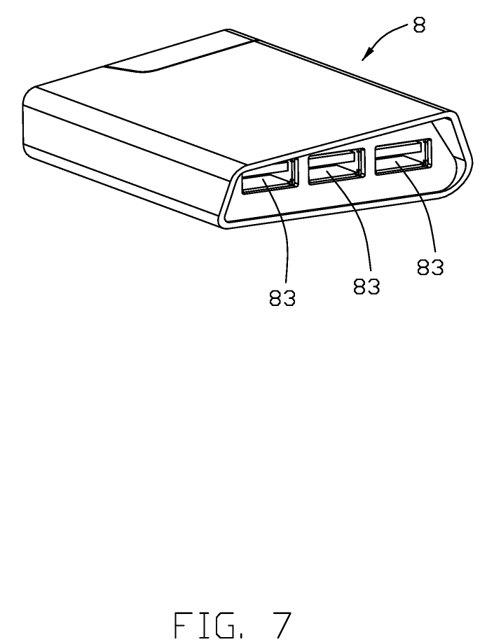

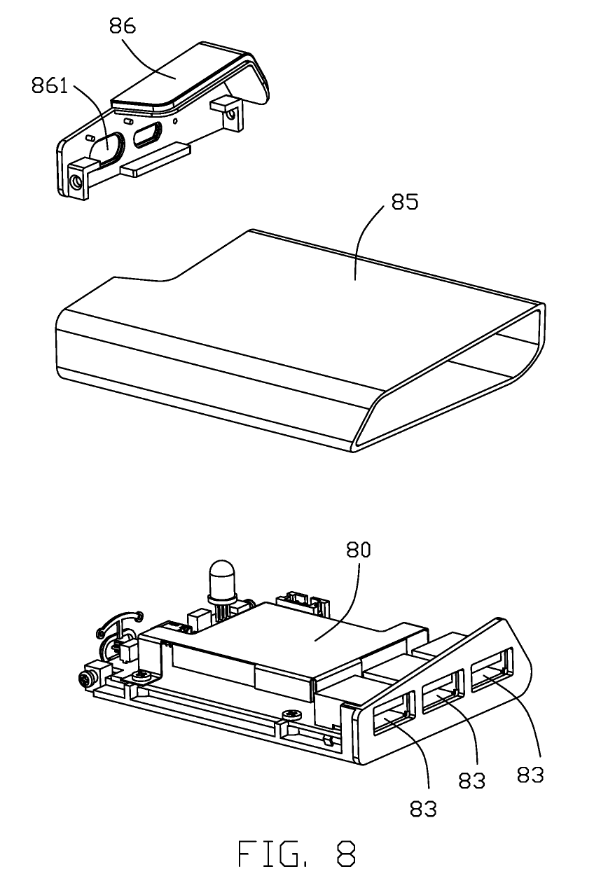

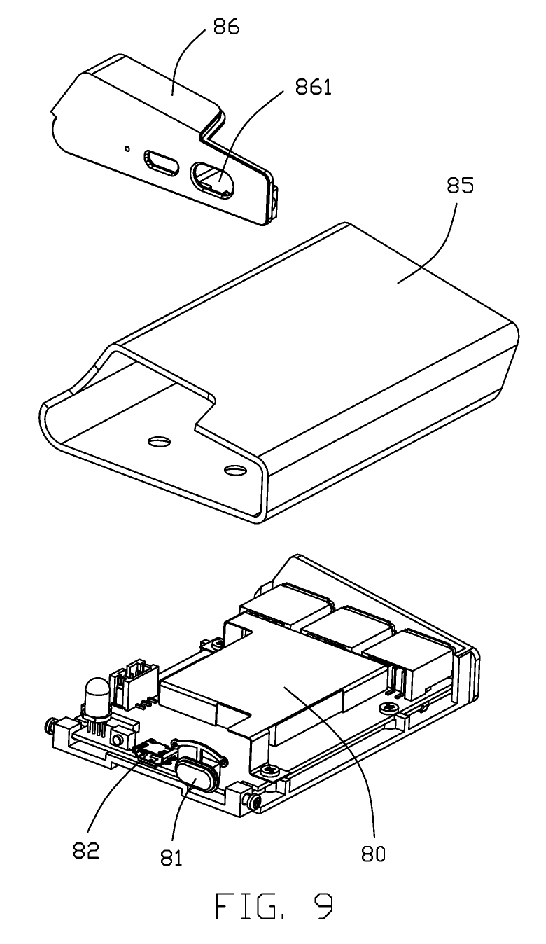

Referring to FIGS. 6-9, the data collection box 8 can be located on the front side of the treadmill and includes on one side a power key 81 for on/off usage, a Micro USB connection port 82, and on the other side three USB connection ports 83 for collecting the data from the sensors 1. The data collection box 8 further includes a printed circuit board assembly (PCBA) 80, an external shell 83 to cover the printed circuit board assembly 80, and a cover 86 upon one side of the external shell 83. The PCBA 80 may manage the data, the power key 81 and the Micro USB connection port 82 are located by one side of the PCBA 80, the cover 86 forms the opening 861 to expose the power key 81 and the Micro USB 82, while the USB connection ports 83 are located by the other side of the PCBA 80.

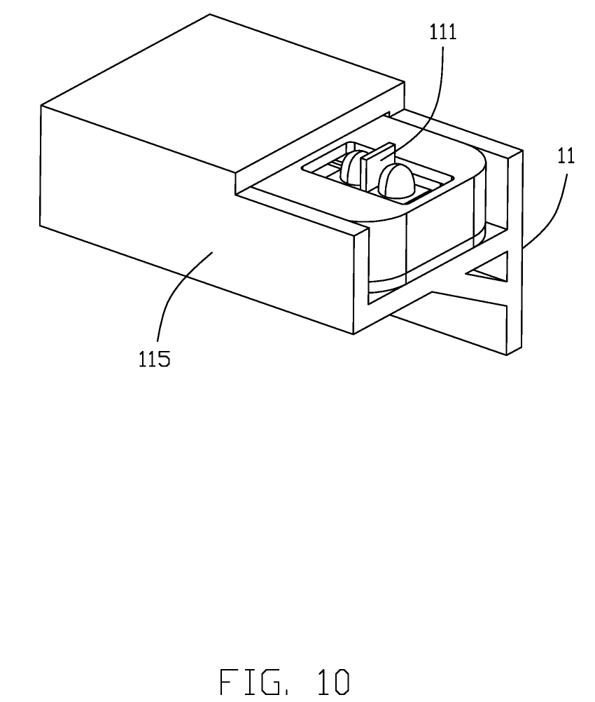

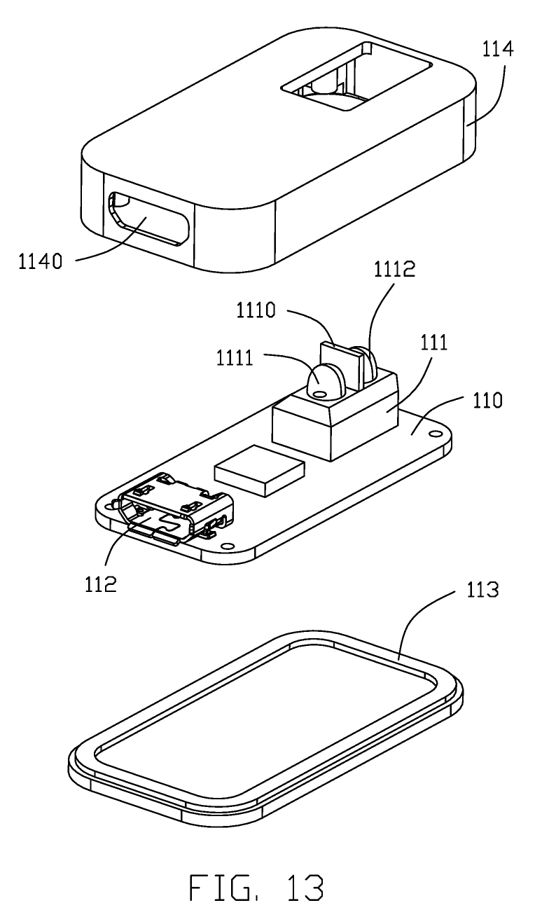

Referring to FIGS. 10 and 13, the infrared sensor 11 includes a first printed circuit board (PCB) 110, the infrared reflection module 111 mounted thereon. A first Micro USB connection port 112 is assembled upon the first PCB 110. A bottom cover 113 and the top cover 114 commonly enclose the first PCB 110. A mounting securing module 115 encloses the bottom 113 and the top cover 114. The top cover 114 includes an opening 1140 for exposing the first Micro USB connection port 112 for connection with the USB connection port 82 via a USB/Micro USB adaptor wire 14. The infrared reflection module 111 is exposed outside and includes an emitter (not labeled), a receiver (not labeled) and a divider 1110. During operation, the lighting piece 1111 of the emitter emits the light and the receiving piece 1112 receives the light. The reflected light may induce the minor signal.

As shown in FIG. 6, the infrared sensor 11 is mounted under the platform 101, and the track belt 102 is equipped with a reflection tape 103 thereon. When the belt 102 takes one cycle, the infrared sensor 11 may experience twice reflection signals, thus obtaining the consuming time. By cooperation with the already known length of the belt 102, the speed of the belt 102 can be know. At the same time, the infrared module 111 knows the amount of the running cycle so as to know the total running distance.

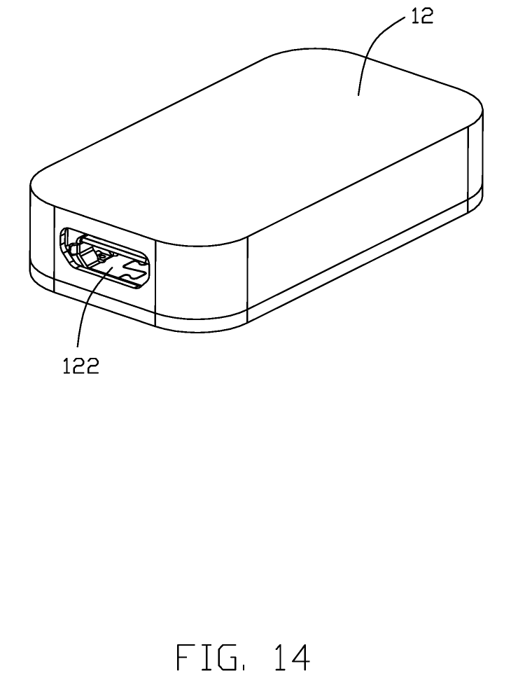

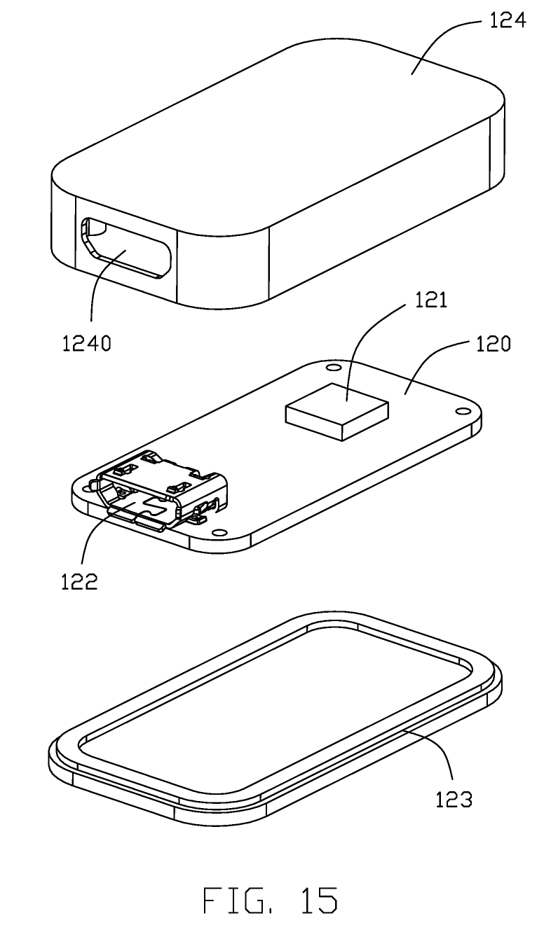

Referring to FIGS. 14-15, the six axes sensor 12 includes a second PCB 120, the processing module 121 mounted thereon. A second Micro USB connection port 122 is assembled upon the second PCB 120. A bottom cover 123 and a top cover 124 commonly enclose the second PCB 120. The processing module 121 includes the accelerometer and the gyroscope. The top cover 124 forms an opening 1240 for exposing the second Micro USB connection port 122 to connect to the USB connection port 82 of the data collection box 8 via the Micro USB and USB wherein the second Micro USB connection port is connected to the USB connection port of the data collection box via the USB/Micro USB wire adaptor 14. The six axes sensor 12 is located under the platform 101. The accelerometer may measure the slope of the track belt. The signal of step frequency measured by the accelerometer and the gyroscope may be transmitted to the data collection box 8 via the wire 14 so that the step frequency can be obtained via the PCBA 80.

Notably, the traditional treadmill provides the electrocardiogram while the information is only shown on the display of the treadmill. Referring to FIG. 6, in the invention an electrocardiogram sensor 13 is provided and includes electrode plates 130, the wires 131 and the connector (not labeled). The electrode plate 130 is mounted upon the metal handle 104 so as to have the heartbeat signal transmitted to the data collection box 8.

Referring to FIG. 1, the gateway 3 and the data collection box 8 are connected to and mutually communicate with each other via WPAN (Wireless Personal Area Network), e.g. the blue tooth. The data collected from the sensor 1 via the USB wire 14 or the wireless can be managed in the data collection box 8 and transmitted to the gateway 3.

The gateway 3 and the Wi-Fi wireless network station 5 communicate with each other via LAN (Local Area Network) or WAN (Wide Area Network), e.g., the Internet. The station 5 may include the Wi-Fi module and the wireless network station (hot spot or access point).

Referring to FIG. 1, the Wi-Fi wireless network station 5 communicate with the server system 6 via LAN or WAN. The server system 6 includes the web server 61 and the data server 62. The web server 61 includes RFID 4 for identifying the user's personal information for log-in use and the corresponding personal data collected from the sensor 1 and browsing. The data server 62 may store all the data of all users and all data of all machines. The server system 6 may transmit, via the WAN, the data derived from the user, the treadmill, the intelligent power 2 and the room circumstance sensor 7, to either the browsers or the application programs of the mobile device.

The server system 6 may provide partly the DMZ (Demilitarized Zone) or perimeter network 63 for limiting the approaching from the exterior to the enterprise server system.

The gateway 3 communicates with the data collection box 8, the intelligent power 2, the room circumstance sensor 7 and the Wi-Fi wireless network station 5. The gateway 3 transmit the data under different protocols so as to have the data derived from the data collection box 8, the intelligent power 2 and the room circumstance sensor 7 all directed toward the gateway 3 for integrating.

The communication between the data collection box 8 and the sensor 1, and those between the gateway 3 and the data collection box 8/the intelligent power 2/the room circumstance sensor 7 via PAN while the communication between the gateway 3, the RFID 4, the Wi-Fi wireless network station 5 and the server system 6 can be implemented via LAN and WAN.

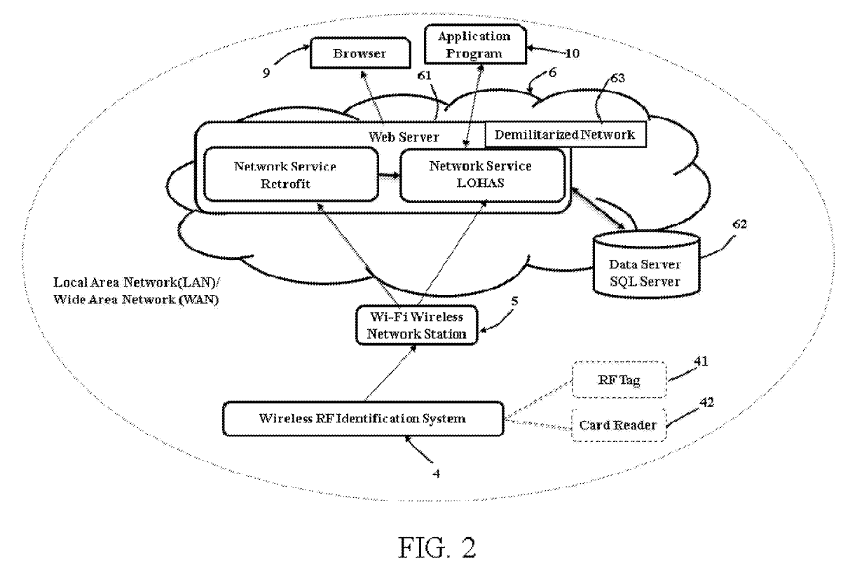

Referring to FIG. 2, the RFID 4 includes the tag 41 corresponding the individual user, and the (card) reader 42 corresponding to the individual treadmill. The reader 42 is connected with the Wi-Fi wireless network station 5. When the user is using the machine, the tag 41 communicates with the reader 42 so the data is transmitted to the wireless network station and further to the web server 61 via LAN or WAN. The web server 61 cooperates with the data server 62 to obtain the stored corresponding data for further reviewing via the browser or the application programs. The all system not only identifies the user but also retrieve the old data and store the new data of the user for efficiently and intelligently managing the data for superior exercise.

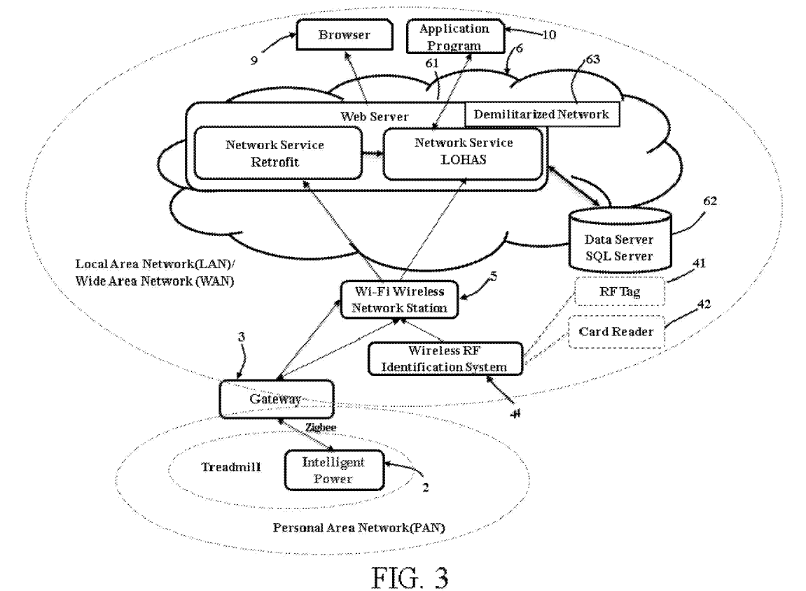

Referring to FIG. 3, the intelligent power 2 mutually communicates with the gateway 3 via low Rate Wireless Personal Area Network (LR-WPAN), e.g., ZigBee.

Referring to FIGS. 1 and 3, when the RFID 4 transmits the data to the gateway 3 via the Wi-Fi wireless network station, the gateway 3 may activate the intelligent power ready in use for saving power in the regular time.

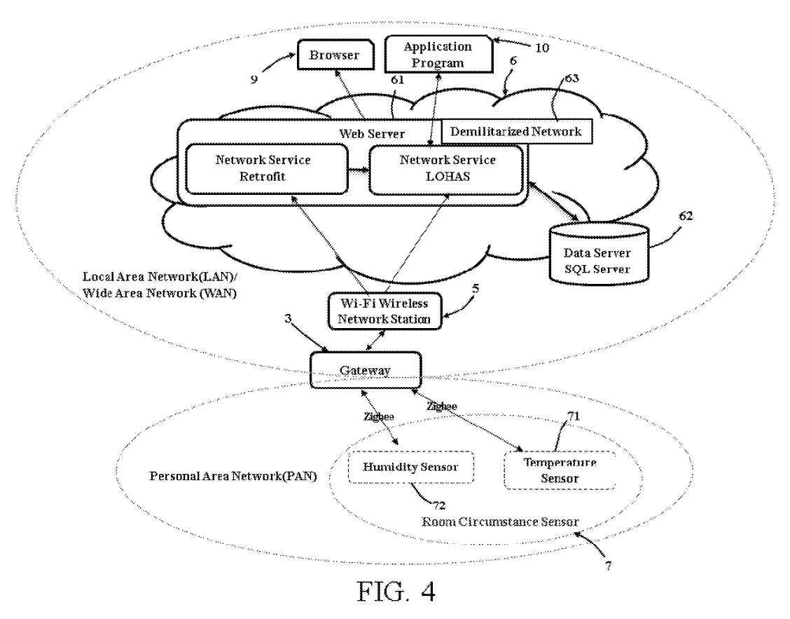

FIG. 4 shows the gateway 3 and the room circumstance sensor 7 mutually communicate with each other via LR-WPAN, e.g. ZigBee. The circumstance sensors 7 may include the temperature sensor 71 and the humidity sensor 72. The room circumstance sensor 7 may transmit the related data to the gateway 3 via ZigBee and further to the Wi-Fi wireless network station and finally to the server system 6 so as to allow the user or the manager/coach to view the related data via the browser 9 or the application program 10. Therefore, the room circumstance may be timely adjusted, if necessary.

Referring to FIG. 5, when the user is using the machine, the infrared sensor 11, the six axes sensor 12 and the electrocardiogram sensor 13 may obtain transmit the obtained data reflecting the heartbeat, the step frequency, the speed, the slope of the belt, the total running distance, to the data collection box 8 via the blue tooth or wires 14. The data collection box manages the collected data and transfer the corresponding data to the gateway 3 via blue tooth. The gateway 3 further transmits the data to the Wi-Fi wireless network station 5 and finally to the server 6 to allow the user or the manager to view the data in time either via the browser 9 or the app 10. Therefore, via this monitoring/computing, the user and the treadmill may interactively join each other for superior adjustment and control.

In conclusion, the data collection box 8 may collect the related data from the sensors 1 via either the wires 14 or the blue tooth and further transfer the computed data to the gateway 3. The invention also uses the gateway 3 to integrate the data collection box 8, the intelligent power 2, and the room circumstance sensor 7, which are under the different protocols, together. The Wi-Fi wireless network station 5 also collects the personal data via RFID 4 and the computed data from the gateway 3 to the server system 6 for efficient management.

However, the disclosure is illustrative only, changes may be made in detail, especially in matter of shape, size, and arrangement of sections within the principles of the invention.

What is claimed is:

1. An intelligent sensing and computing system for use with a treadmill, comprising:
a gateway and an RFID (Radio Frequency Identification) commonly connected to a Wi-Fi wireless network station;
a server system connected to the Wi-Fi wireless network station;
a data collection box connected to the gateway; and
sensors for sensing a heartbeat of a user, a speed and a slope of the treadmill connected to the data collection box.
